SEQUENCE LISTING (134 sequences)

SEQ ID NO: 1 - protein, 17 aa, Artificial sequence
  Synthetic peptide.
GEGQQHHLGGAKQAGDV

SEQ ID NO: 2 - protein, 17 aa, Artificial sequence
  Synthetic peptide.
GEGQQHHLGGAAQAGDV

SEQ ID NO: 3 - protein, 17 aa, Artificial sequence
  synthetic peptide
GEGQQHHLGGAKQAGAV

SEQ ID NO: 4 - protein, 15 aa, Artificial sequence
  synthetic peptide
GQQHHLGGAKQAGDV

SEQ ID NO: 5 - protein, 15 aa, Artificial sequence
  synthetic peptide
GQQHHLGGAKKAGDV

SEQ ID NO: 6 - protein, 17 aa, Artificial sequence
  synthetic peptide
GEGQQHHLGGAKKAGDV

SEQ ID NO: 7 - protein, 17 aa, Artificial sequence
  synthetic peptide
GEGQQHHLGGAARAGDV

SEQ ID NO: 8 - protein, 17 aa, Artificial sequence
  synthetic peptide
GEGQQHHLGGAKHAGDV

SEQ ID NO: 9 - protein, 15 aa, Artificial sequence
  synthetic peptide
NEEGFFSARGHRPLD

SEQ ID NO: 10 - protein, 5 aa, Artificial sequence
  synthetic peptide
FFSAR

SEQ ID NO: 11 - protein, 5 aa, Artificial sequence
  synthetic peptide
GGGAR

SEQ ID NO: 12 - protein, 11 aa, Artificial sequence
  synthetic peptide
GGGVRGPRVVE

SEQ ID NO: 13 - protein, 11 aa, Artificial sequence
  synthetic peptide
GFFSARGHRPL

SEQ ID NO: 14 - protein, 11 aa, Artificial sequence
  synthetic peptide
GFFGARGHRPL

SEQ ID NO: 15 - protein, 11 aa, Artificial sequence
  synthetic peptide
GFGGARGHRPL

SEQ ID NO: 16 - protein, 11 aa, Artificial sequence
  synthetic peptide
GGGGARGHRPL

SEQ ID NO: 17 - protein, 11 aa, Artificial sequence
  synthetic peptide
GGGGVRGHRPL

SEQ ID NO: 18 - protein, 8 aa, Artificial sequence
  synthetic peptide
LDPRIVDG

SEQ ID NO: 19 - protein, 8 aa, Artificial sequence
  synthetic peptide
VDPRLIDG

SEQ ID NO: 20 - protein, 11 aa, Artificial sequence
  synthetic peptide
GFFDARGHRPL

SEQ ID NO: 21 - protein, 11 aa, Artificial sequence
  synthetic peptide
GFGDARGHRPL

SEQ ID NO: 22 - protein, 11 aa, Artificial sequence
  synthetic peptide
GGGDARGHRPL

SEQ ID NO: 23 - protein, 11 aa, Artificial sequence
  synthetic peptide
GGGDVRGHRPL

SEQ ID NO: 24 - protein, 17 aa, Artificial sequence
  synthetic peptide
GEGQQGHLGGAKQAGDV

SEQ ID NO: 25 - protein, 17 aa, Artificial sequence
  synthetic peptide
GEGQQAHLGGAKQAGDV

SEQ ID NO: 26 - protein, 17 aa, Artificial sequence
  synthetic peptide
GEGQQVHLGGAKQAGDV

SEQ ID NO: 27 - protein, 17 aa, Artificial sequence
  synthetic peptide
GEGQQLHLGGAKQAGDV

SEQ ID NO: 28 - protein, 17 aa, Artificial sequence
  synthetic peptide
GEGQQIHLGGAKQAGDV

SEQ ID NO: 29 - protein, 17 aa, Artificial sequence
  synthetic peptide
GEGQQMHLGGAKQAGDV

SEQ ID NO: 30 - protein, 17 aa, Artificial sequence
  synthetic peptide
GEGQQPHLGGAKQAGDV

SEQ ID NO: 31 - protein, 17 aa, Artificial sequence
  synthetic peptide
GEGQQFHLGGAKQAGDV

SEQ ID NO: 32 - protein, 17 aa, Artificial sequence
  synthetic peptide
GEGQQWHLGGAKQAGDV

SEQ ID NO: 33 - protein, 17 aa, Artificial sequence
  synthetic peptide
GEGQQHLLGGAKQAGDV

SEQ ID NO: 34 - protein, 17 aa, Artificial sequence
  synthetic peptide
GEGQQHMLGGAKQAGDV

SEQ ID NO: 35 - protein, 17 aa, Artificial sequence
  synthetic peptide
GEGQQHCLGGAKQAGDV

SEQ ID NO: 36 - protein, 17 aa, Artificial sequence
  synthetic peptide
GEGQQHPLGGAKQAGDV

SEQ ID NO: 37 - protein, 17 aa, Artificial sequence
  synthetic peptide
GEGQQHHEGGAKQAGDV

SEQ ID NO: 38 - protein, 17 aa, Artificial sequence
  synthetic peptide
GEGQQHHKGGAKQAGDV

SEQ ID NO: 39 - protein, 17 aa, Artificial sequence
  synthetic peptide
GEGQQHHRGGAKQAGDV

SEQ ID NO: 40 - protein, 17 aa, Artificial sequence
  synthetic peptide
GEGQQHHMGGAKQAGDV

SEQ ID NO: 41 - protein, 17 aa, Artificial sequence
  synthetic peptide
GEGQQHHFGGAKQAGDV

SEQ ID NO: 42 - protein, 17 aa, Artificial sequence
  synthetic peptide
GEGQQHHWGGAKQAGDV

SEQ ID NO: 43 - protein, 17 aa, Artificial sequence
  synthetic peptide
GEGQQHHLIGAKQAGDV

SEQ ID NO: 44 - protein, 17 aa, Artificial sequence
  synthetic peptide
GEGQQHHLQGAKQAGDV

SEQ ID NO: 45 - protein, 17 aa, Artificial sequence
  synthetic peptide
GEGQQHHLEGAKQAGDV

SEQ ID NO: 46 - protein, 17 aa, Artificial sequence
  synthetic peptide
GEGQQHHLMGAKQAGDV

SEQ ID NO: 47 - protein, 17 aa, Artificial sequence
  synthetic peptide
GEGQQHHLCGAKQAGDV

SEQ ID NO: 48 - protein, 17 aa, Artificial sequence
  synthetic peptide
GEGQQHHLPGAKQAGDV

SEQ ID NO: 49 - protein, 17 aa, Artificial sequence
  synthetic peptide
GEGQQHHLFGAKQAGDV

SEQ ID NO: 50 - protein, 17 aa, Artificial sequence
  synthetic peptide
GEGQQHHLWGAKQAGDV

SEQ ID NO: 51 - protein, 17 aa, Artificial sequence
  synthetic peptide
GEGQQHHLGAAKQAGDV

SEQ ID NO: 52 - protein, 17 aa, Artificial sequence
  synthetic peptide
GEGQQHHLGVAKQAGDV

SEQ ID NO: 53 - protein, 17 aa, Artificial sequence
  synthetic peptide
GEGQQHHLGLAKQAGDV

SEQ ID NO: 54 - protein, 17 aa, Artificial sequence
  synthetic peptide
GEGQQHHLGIAKQAGDV

SEQ ID NO: 55 - protein, 17 aa, Artificial sequence
  synthetic peptide
GEGQQHHLGTAKQAGDV

SEQ ID NO: 56 - protein, 17 aa, Artificial sequence
  synthetic peptide
GEGQQHHLGNAKQAGDV

SEQ ID NO: 57 - protein, 17 aa, Artificial sequence
  synthetic peptide
GEGQQHHLGQAKQAGDV

SEQ ID NO: 58 - protein, 17 aa, Artificial sequence
  synthetic peptide
GEGQQHHLGDAKQAGDV

SEQ ID NO: 59 - protein, 17 aa, Artificial sequence
  synthetic peptide
GEGQQHHLGEAKQAGDV

SEQ ID NO: 60 - protein, 17 aa, Artificial sequence
  synthetic peptide
GEGQQHHLGHAKQAGDV

SEQ ID NO: 61 - protein, 17 aa, Artificial sequence
  synthetic peptide
GEGQQHHLGKAKQAGDV

SEQ ID NO: 62 - protein, 17 aa, Artificial sequence
  synthetic peptide
GEGQQHHLGRAKQAGDV

SEQ ID NO: 63 - protein, 17 aa, Artificial sequence
  synthetic peptide
GEGQQHHLGMAKQAGDV

SEQ ID NO: 64 - protein, 17 aa, Artificial sequence
  synthetic peptide
GEGQQHHLGCAKQAGDV

SEQ ID NO: 65 - protein, 17 aa, Artificial sequence
  synthetic peptide
GEGQQHHLGYAKQAGDV

SEQ ID NO: 66 - protein, 17 aa, Artificial sequence
  synthetic peptide
GEGQQHHLGFAKQAGDV

SEQ ID NO: 67 - protein, 17 aa, Artificial sequence
  synthetic peptide
GEGQQHHLGWAKQAGDV

SEQ ID NO: 68 - protein, 17 aa, Artificial sequence
  synthetic peptide
GEGQQHHLGGLKQAGDV

SEQ ID NO: 69 - protein, 17 aa, Artificial sequence
  synthetic peptide
GEGQQHHLGGNKQAGDV

SEQ ID NO: 70 - protein, 17 aa, Artificial sequence
  synthetic peptide
GEGQQHHLGGQKQAGDV

SEQ ID NO: 71 - protein, 17 aa, Artificial sequence
  synthetic peptide
GEGQQHHLGGDKQAGDV

SEQ ID NO: 72 - protein, 17 aa, Artificial sequence
  synthetic peptide
GEGQQHHLGGEKQAGDV

SEQ ID NO: 73 - protein, 17 aa, Artificial sequence
  synthetic peptide
GEGQQHHLGGHKQAGDV

SEQ ID NO: 74 - protein, 17 aa, Artificial sequence
  synthetic peptide
GEGQQHHLGGKKQAGDV

SEQ ID NO: 75 - protein, 17 aa, Artificial sequence
  synthetic peptide
GEGQQHHLGGRKQAGDV

SEQ ID NO: 76 - protein, 17 aa, Artificial sequence
  synthetic peptide
GEGQQHHLGGMKQAGDV

SEQ ID NO: 77 - protein, 17 aa, Artificial sequence
  synthetic peptide
GEGQQHHLGGYKQAGDV

SEQ ID NO: 78 - protein, 17 aa, Artificial sequence
  synthetic peptide
GEGQQHHLGGFKQAGDV

SEQ ID NO: 79 - protein, 17 aa, Artificial sequence
  synthetic peptide
GEGQQHHLGGWKQAGDV

SEQ ID NO: 80 - protein, 17 aa, Artificial sequence
  synthetic peptide
GEGQQHHLGGAKGAGDV

SEQ ID NO: 81 - protein, 17 aa, Artificial sequence
  synthetic peptide
GEGQQHHLGGAKAAGDV

SEQ ID NO: 82 - protein, 17 aa, Artificial sequence
  synthetic peptide
GEGQQHHLGGAKVAGDV

SEQ ID NO: 83 - protein, 17 aa, Artificial sequence
  synthetic peptide
GEGQQHHLGGAKLAGDV

SEQ ID NO: 84 - protein, 17 aa, Artificial sequence
  synthetic peptide
GEGQQHHLGGAKIAGDV

SEQ ID NO: 85 - protein, 17 aa, Artificial sequence
  synthetic peptide
GEGQQHHLGGAKSAGDV

SEQ ID NO: 86 - protein, 17 aa, Artificial sequence
  synthetic peptide
GEGQQHHLGGAKTAGDV

SEQ ID NO: 87 - protein, 17 aa, Artificial sequence
  synthetic peptide
GEGQQHHLGGAKNAGDV

SEQ ID NO: 88 - protein, 17 aa, Artificial sequence
  synthetic peptide
GEGQQHHLGGAKDAGDV

SEQ ID NO: 89 - protein, 17 aa, Artificial sequence
  synthetic peptide
GEGQQHHLGGAKEAGDV

SEQ ID NO: 90 - protein, 17 aa, Artificial sequence
  synthetic peptide
GEGQQHHLGGAKMAGDV

SEQ ID NO: 91 - protein, 17 aa, Artificial sequence
  synthetic peptide
GEGQQHHLGGAKCAGDV

SEQ ID NO: 92 - protein, 17 aa, Artificial sequence
  synthetic peptide
GEGQQHHLGGAKPAGDV

SEQ ID NO: 93 - protein, 17 aa, Artificial sequence
  synthetic peptide
GEGQQHHLGGAKYAGDV

SEQ ID NO: 94 - protein, 17 aa, Artificial sequence
  synthetic peptide
GEGQQHHLGGAKFAGDV

SEQ ID NO: 95 - protein, 17 aa, Artificial sequence
  synthetic peptide
GEGQQHHLGGAKWAGDV

SEQ ID NO: 96 - protein, 17 aa, Artificial sequence
  synthetic peptide
GEGQQHHLGGAKQVGDV

SEQ ID NO: 97 - protein, 17 aa, Artificial sequence
  synthetic peptide
GEGQQHHLGGAKQLGDV

SEQ ID NO: 98 - protein, 17 aa, Artificial sequence
  synthetic peptide
GEGQQHHLGGAKQIGDV

SEQ ID NO: 99 - protein, 17 aa, Artificial sequence
  synthetic peptide
GEGQQHHLGGAKQSGDV

SEQ ID NO: 100 - protein, 17 aa, Artificial sequence
  synthetic peptide
GEGQQHHLGGAKQTGDV

SEQ ID NO: 101 - protein, 17 aa, Artificial sequence
  synthetic peptide
GEGQQHHLGGAKQNGDV

SEQ ID NO: 102 - protein, 17 aa, Artificial sequence
  synthetic peptide
GEGQQHHLGGAKQQGDV

SEQ ID NO: 103 - protein, 17 aa, Artificial sequence
  synthetic peptide
GEGQQHHLGGAKQDGDV

SEQ ID NO: 104 - protein, 17 aa, Artificial sequence
  synthetic peptide
GEGQQHHLGGAKQEGDV

SEQ ID NO: 105 - protein, 17 aa, Artificial sequence
  synthetic peptide
GEGQQHHLGGAKQHGDV

SEQ ID NO: 106 - protein, 17 aa, Artificial sequence
  synthetic peptide
GEGQQHHLGGAKQKGDV

SEQ ID NO: 107 - protein, 17 aa, Artificial sequence
  synthetic peptide
GEGQQHHLGGAKQRGDV

SEQ ID NO: 108 - protein, 17 aa, Artificial sequence
  synthetic peptide
GEGQQHHLGGAKQMGDV

SEQ ID NO: 109 - protein, 17 aa, Artificial sequence
  synthetic peptide
GEGQQHHLGGAKQCGDV

SEQ ID NO: 110 - protein, 17 aa, Artificial sequence
  synthetic peptide
GEGQQHHLGGAKQPGDV

SEQ ID NO: 111 - protein, 17 aa, Artificial sequence
  synthetic peptide
GEGQQHHLGGAKQYGDV

SEQ ID NO: 112 - protein, 17 aa, Artificial sequence
  synthetic peptide
GEGQQHHLGGAKQFGDV

SEQ ID NO: 113 - protein, 17 aa, Artificial sequence
  synthetic peptide
GEGQQHHLGGAKQWGDV

SEQ ID NO: 114 - protein, 17 aa, Artificial sequence
  synthetic peptide
GEGQQHHLGGAKQAADV

SEQ ID NO: 115 - protein, 17 aa, Artificial sequence
  synthetic peptide
GEGQQHHLGGAKQAVDV

SEQ ID NO: 116 - protein, 17 aa, Artificial sequence
  synthetic peptide
GEGQQHHLGGAKQALDV

SEQ ID NO: 117 - protein, 17 aa, Artificial sequence
  synthetic peptide
GEGQQHHLGGAKQAIDV

SEQ ID NO: 118 - protein, 17 aa, Artificial sequence
  synthetic peptide
GEGQQHHLGGAKQASDV

SEQ ID NO: 119 - protein, 17 aa, Artificial sequence
  synthetic peptide
GEGQQHHLGGAKQATDV

SEQ ID NO: 120 - protein, 17 aa, Artificial sequence
  synthetic peptide
GEGQQHHLGGAKQANDV

SEQ ID NO: 121 - protein, 17 aa, Artificial sequence
  synthetic peptide
GEGQQHHLGGAKQAQDV

SEQ ID NO: 122 - protein, 17 aa, Artificial sequence
  synthetic peptide
GEGQQHHLGGAKQADDV

SEQ ID NO: 123 - protein, 17 aa, Artificial sequence
  synthetic peptide
GEGQQHHLGGAKQAEDV

SEQ ID NO: 124 - protein, 17 aa, Artificial sequence
  synthetic peptide
GEGQQHHLGGAKQAHDV

SEQ ID NO: 125 - protein, 17 aa, Artificial sequence
  synthetic peptide
GEGQQHHLGGAKQAKDV

SEQ ID NO: 126 - protein, 17 aa, Artificial sequence
  synthetic peptide
GEGQQHHLGGAKQARDV

SEQ ID NO: 127 - protein, 17 aa, Artificial sequence
  synthetic peptide
GEGQQHHLGGAKQAMDV

SEQ ID NO: 128 - protein, 17 aa, Artificial sequence
  synthetic peptide
GEGQQHHLGGAKQACDV

SEQ ID NO: 129 - protein, 17 aa, Artificial sequence
  synthetic peptide
GEGQQHHLGGAKQAPDV

SEQ ID NO: 130 - protein, 17 aa, Artificial sequence
  synthetic peptide
GEGQQHHLGGAKQAYDV

SEQ ID NO: 131 - protein, 17 aa, Artificial sequence
  synthetic peptide
GEGQQHHLGGAKQAFDV

SEQ ID NO: 132 - protein, 17 aa, Artificial sequence
  synthetic peptide
GEGQQHHLGGAKQAWDV

SEQ ID NO: 133 - protein, 28 aa, Artificial sequence
  synthetic peptide
MKIIPFNRLTIGEGQQHHLGGAKQAGDV

SEQ ID NO: 134 - protein, 29 aa, Artificial sequence
  synthetic peptide
MKIIPTNRITAGDGQQTGGVKQFGGLGDN

(19) United States
(12) Patent Application Publication
(10) Pub. No.: US 2011/0171285 A1
(43) Pub. Date: Jul. 14, 2011

(54) Design of Fibrinogen and Fibrinogen Derived Products with Reduced Bacterial Binding by Using Modified Sequences of Fibrinogen Chains

(75) Inventors: MAGNUS HOOK, HOUSTON, TX (US); VANNAKAMBADI K. GANESH, PEARLAND, TX (US); EMANUEL SMEDS, HOUSTON, TX (US)

(73) Assignee: THE TEXAS A&M UNIVERSITY SYSTEM, COLLEGE STATION, TX (US)

(21) Appl. No.: 12/914,511

(22) Filed: Oct. 28, 2010

Related U.S. Application Data
(60) Provisional application No. 61/255,806, filed on Oct. 28, 2009.

(51) Int. Cl. A61K 38/48; C07K 14/435; A61K 8/64; A61L 15/32; A61Q 17/04; A61P 17/02; A61P 7/04; A61P 31/00; A61P 31/12; A61P 31/10; A61P 29/00; A61P 23/02; A61K 9/14; A61K 9/00; C07H 21/04; C12N 15/63

(52) U.S. Cl. 424/447; 530/380; 424/94.64; 424/59; 514/13.5; 424/484; 424/400; 514/13.8; 514/14.9; 536/23.5; 435/320.1

(57) ABSTRACT

The present invention describes the design and development of new fibrinogen and fibrinogen derived products with significantly reduced binding to bacteria while retaining normal physiological functions by using modified fibrinogen amino acid sequences. The present invention describes modified sequences of Fg γ-chains and β-chains with reduced binding to S. aureus ClfA and S. epidermidis SdrG respectively. Modified Fg with the described modifications will not bind other bacterial surface proteins that bind Fg using similar mechanisms as ClfA and SdrG. These new Fg and Fg derived products will therefore have less binding to bacteria and will be advantageous compared to normal human Fg in a number of different settings.

### CROSS-REFERENCE TO RELATED APPLICATIONS

This application claims priority to U.S. Provisional Application Ser. No. 61/255,806, filed Oct. 28, 2009, the contents of which is incorporated by reference herein in its entirety.

### STATEMENT OF FEDERALLY FUNDED RESEARCH

This invention was made with U.S. Government support under Contract No. AI20624 awarded by the National Institutes of Health (NIH). The government has certain rights in this invention.

### SEQUENCE LISTING

Sequence listing submitted separately.

### INCORPORATION-BY-REFERENCE OF MATERIALS FILED ON COMPACT DISC

Not Applicable.

### TECHNICAL FIELD OF THE INVENTION

The present invention relates in general to the field of synthetic or isolated proteins, and more specifically to the development and/or characterization of fibrinogen sequences comprising modified beta- and/or gamma-chains to make fibrinogen products with reduced bacterial adhesion while retaining normal physiological functions.

### BACKGROUND OF THE INVENTION

Without limiting the scope of the invention, its background is described in connection with recombinant and modified fibrinogens and bacterial adhesion to fibrinogen and related products and methods for preventing the same.

U.S. Pat. No. 6,177,084 issued to Foster and Mcdevitt (2001) describes the isolation of the S. aureus fibrinogen binding protein gene and a minimal fibrinogen binding protein is identified. The protein as described in the '084 patent finds use as a vaccine or a pharmaceutical composition for application to prevent infection, promotion of wound healing, blocking adherence to indwelling medical devices, or diagnosis of infection.

U.S. Pat. No. 6,037,457 issued to Lord (2000) discloses a method of producing recombinant fibrinogen in a long-term mammalian cell culture system. The method of the '457 patent for the production of recombinant fibrinogen, comprises: growing mammalian cells that express recombinant fibrinogen in a serum-free medium for a time of at least one month at a level of at least 5 μg/ml; and then collecting portions of the conditioned medium at least twice during the culturing time of at least one month, with each portion containing at least 5 μg/ml of recombinant fibrinogen. Also disclosed is a method for the production of recombinant fibrinogen, comprising: growing mammalian cells that express recombinant fibrinogen in a serum-free medium at a level greater than 1 μg/ml; collecting at least a portion of the conditioned medium, and then purifying the fibrinogen from the medium by anion-exchange chromatography or affinity chromatography. In some embodiments of the invention, the medium is concentrated prior to the step of purifying the fibrinogen. In alternate embodiments, both the concentrating and purifying steps are carried out in the presence of at least one protease inhibitor.

U.S. Patent Application Ser. No. 61/133,537 discloses crystal structure of Staphylococcus aureus Clumping factor A (ClfA) in complex with fibrinogen (Fg) derived peptide. The present invention also discloses the use of this structure in the design of ClfA targeted vaccines and therapeutic agents (including monoclonal antibodies). In addition, the present invention discloses isolated and purified engineered Staphylococcus clumping factor A protein (ClfA) with a stabilized, closed conformation and immunogenic compositions thereof including methods of treating a Staphylococcus infection in an individual.

### BRIEF SUMMARY OF THE INVENTION

The present invention describes compositions, methods and uses for substituted fibrinogen (Fg) amino acid sequences to make new fibrinogen products that will have significantly reduced binding to bacteria. These new products prevent bacterial attachment or even treat conditions resulting from mild or serious bacterial infections. Treatment with this modified Fg may be useful wherever normal Fg is currently used, including systemic use or localized application in patients. The modified Fg may be used in applications like fibrin glue or fibrin sealant, coated to medical devices or systemically administered to patients with certain medical conditions. Further, the modified Fg and Fg derived products can also be made with a reduced binding to platelets and used to coat medical devices. The invention also lists possible hybrid molecules of bacterial Fg-binding molecules that are linked to modified fibrinogen β- and γ-chain sequences that will block bacterial binding but can retain host functions.

The present invention describes a recombinant, transgenic or isolated natural variant fibrinogen protein comprising a fibrinogen wherein the γ-chain comprises one or more modified 17 C-terminal residues. The sequence of the modified 17 C-terminal residues is selected from SEQ. ID NO: 6, SEQ. ID NO: 7 or SEQ. ID NO: 8. The fibrinogen protein of the present invention is a human fibrinogen protein and is adapted for therapeutic use as a nasal spray, a foam, an eye drop, or a topical wound dressing in a pharmaceutically acceptable excipient. In one aspect of the present invention the fibrinogen protein is non-ovine. In another aspect the fibrinogen β-chain comprises an N-terminal modified β-chain, wherein the sequence of the N-terminal modified β-chains is selected from SEQ. ID NO: 14, SEQ. ID NO: 15, SEQ. ID NO: 16, SEQ. ID NO: 17, SEQ. ID NO: 20, SEQ. ID NO: 21, SEQ. ID NO: 22 or SEQ. ID NO: 23. In yet another aspect the present invention describes a recombinant, transgenic or natural variant fibrinogen protein comprising a non-ovine fibrinogen and an ovine γ-chain sequence. In addition the sequences SEQ ID NO: 24-132 are also included herein and may be used in the present invention.

The present invention also includes a fibrin glue or sealant composition comprising, (i) a first container comprising a fibrinogen with a γ-chain comprising one or more modified 17 C-terminal residues, wherein the sequence of the modified 17 C-terminal residues is selected from SEQ. ID NO: 6, SEQ. ID NO: 7 or SEQ. ID NO: 8 and (ii) a second container comprising a thrombin or one or more proteinases or enzymes, wherein a combination of the contents of the first and second containers form a fibrin clot. The one or more proteinases are selected from the group consisting of batroxobin, okinaxobin, flavoxobin, arvin, cathepsin, or any combinations thereof. In one aspect the fibrinogen is non-ovine and is a human fibrinogen protein. In another aspect the composition further comprises coagulation Factor XIII and an antifibrinolytic agent like ε-aminocaproic acid, p-aminomethylbenzoic acid or aprotinin. In addition the sequences SEQ ID NO: 24-132 are also included herein and may be used in the present invention.

In one embodiment the present invention is a bandage comprising, a backing comprising a paper, a cloth, a plastic, an open-cell polyurethane foam, an open-cell polyethylene foam, a nonwoven fabric and a fibrinogen wherein the γ-chain comprises one or more modified 17 C-terminal residues, wherein the sequence of the modified 17 C-terminal residues is selected from SEQ. ID NO: 6, SEQ. ID NO: 7 or SEQ. ID NO: 8. In one aspect the bandage of the present invention further comprises a carrier selected from a polymer thickener, water, preservatives, active surfactants or emulsifiers, antioxidants, sunscreens, or combinations thereof. In another aspect the bandage further comprises an adhesive or an adhesive layer on the backing. In yet another aspect the carrier further comprises antimicrobial agents, anti-inflammatory agents, antiviral agents, local anesthetic agents, corticosteroids, destructive therapy agents, antifungals, antiandrogens, mild surfactants, and combinations thereof. In a specific aspect the fibrinogen protein is a human fibrinogen protein. In addition the sequences SEQ ID NO: 24-132 are also included herein and may be used in the present invention.

The present invention also discloses a recombinant, transgenic or isolated natural variant fibrinogen protein comprising a fibrinogen wherein a Staphylococcus sp. or a Streptococcus sp. bacterium does not attach to the γ-chain sequence of fibrinogen that comprises a modified 17 C-terminal residues, wherein the sequence of the modified 17 C-terminal residues is selected from SEQ. ID NO: 6, SEQ. ID NO: 7 or SEQ. ID NO: 8. In addition the sequences SEQ ID NO: 24-132 are also included herein and may be used in the present invention.

The present invention also provides for a method of treating wound infections or promoting wound healing comprising the steps of: (i) identifying a patient in need of treatment against wound infections or promotion of wound healing and (ii) applying a fibrin sealant, a fibrin glue or a bandage, wherein the fibrin sealant, the fibrin glue or the bandage comprises a fibrinogen wherein the γ-chain comprises one or more modified 17 C-terminal residues, wherein the sequence of the modified 17 C-terminal residues is selected from SEQ. ID NO: 6, SEQ. ID NO: 7 or SEQ. ID NO: 8. In one aspect of the method of the present invention the fibrinogen protein is a human fibrinogen protein. In another aspect the fibrinogen protein is a non-ovine fibrinogen comprising an ovine γ-chain sequence. In addition, the sequences SEQ ID NO: 24-132 are also included herein and may be used in the present invention.

Another embodiment of the present invention relates to a method of treating sepsis, hypofibrinogenemia or congenital afibrinogenemia in a patient comprising the step of: identifying a patient in need for treatment against sepsis, hypofibrinogenemia or congenital afibrinogenemia and systemically administering a fibrinogen wherein the γ-chain comprises one or more modified 17 C-terminal residues, wherein the sequence of the modified 17 C-terminal residues is selected from SEQ. ID NO: 6, SEQ. ID NO: 7 or SEQ. ID NO: 8. In one aspect of the method of the present invention the fibrinogen protein is a human fibrinogen protein. In another aspect the fibrinogen protein is a non-ovine fibrinogen comprising an ovine γ-chain sequence. In addition the sequences SEQ ID NO: 24-132 are also included herein and may be used in the present invention.

The present invention further describes a biodegradable polymer or matrix composition comprising: a polymer or matrix and a fibrinogen in, on or about the polymer or matrix, wherein the γ-chain comprises one or more modified 17 C-terminal residues, wherein the sequence of the modified 17 C-terminal residues is selected from SEQ. ID NO: 6, SEQ. ID NO: 7 or SEQ. ID NO: 8. In one aspect the fibrinogen protein is a human fibrinogen protein. In another aspect the fibrinogen protein is a non-ovine fibrinogen comprising an ovine γ-chain sequence. In addition the sequences SEQ ID NO: 24-132 are also included herein and may be used in the present invention.

In yet another embodiment the present invention describes a coated medical device, injection device, artificial valve or any medical component in contact with blood comprising a fibrinogen wherein the γ-chain comprises one or more modified 17 C-terminal residues, wherein the sequence of the modified 17 C-terminal residues is selected from SEQ. ID NO: 6, SEQ. ID NO: 7 or SEQ. ID NO: 8. In one aspect the fibrinogen protein is a human fibrinogen protein. In another aspect the fibrinogen protein is a non-ovine fibrinogen comprising an ovine γ-chain sequence. In addition the sequences SEQ ID NO: 24-132 are also included herein and may be used in the present invention.

The present invention further provides for a method of preventing platelet adherence, bacterial adherence on a medical device, an injection device, an artificial valve or any medical component in contact with blood, comprising the step of coating or coupling the medical device, the injection device, the artificial valve or any medical component in contact with blood with a fibrinogen wherein the γ-chain comprises one or more modified 17 C-terminal residues, wherein the sequence of the modified 17 C-terminal residues is selected from SEQ. ID NO: 6, SEQ. ID NO: 7 or SEQ. ID NO: 8. The one or more synthetic or natural variant fibrinogen protein as described in the method of the present invention are coated or coupled by using one or more chemical-cross linkers. In addition the sequences SEQ ID NO: 24-132 are also included herein and may be used in the present invention.

In one embodiment the present invention relates to a method for preventing intravascular thrombosis comprising the step of coating or coupling a medical device, a injection device, a artificial valve or any medical component in contact with blood with a fibrinogen wherein the γ-chain comprises one or more modified 17 C-terminal residues, wherein the sequence of the modified 17 C-terminal residues is selected from SEQ. ID NO: 6, SEQ. ID NO: 7 or SEQ. ID NO: 8. In one aspect of the method of the present invention the coating prevents the adherence of platelets to the medical device, the injection device, the artificial valve or any medical component in contact with blood. In addition the sequences SEQ ID NO: 24-132 are also included herein and may be used in the present invention.

In another embodiment the present invention describes a composition comprising, a fibrinogen wherein the γ-chain comprises one or more modified 17 C-terminal residues, wherein the sequence of the modified 17 C-terminal residues is selected SEQ. ID NO: 6, SEQ. ID NO: 7 or SEQ. ID NO: 8 and one or more optional vaccine adjuvants or excipients. In addition the sequences SEQ ID NO: 24-132 are also included herein and may be used in the present invention.

The present invention describes a pharmaceutical formulation comprising, a recombinant, transgenic or isolated natural variant fibrinogen wherein the γ-chain comprises one or more modified 17 C-terminal residues, wherein the sequence of the modified 17 C-terminal residues is selected from SEQ. ID NO: 6, SEQ. ID NO: 7 or SEQ. ID NO: 8 disposed in one or more pharmaceutically acceptable excipients. In addition the sequences SEQ ID NO: 24-132 are also included herein and may be used in the present invention.

The present invention also describes a nucleic acid comprising a sequence that encodes a recombinant, transgenic or isolated natural variant fibrinogen wherein the γ-chain comprises one or more modified 17 C-terminal residues, wherein the sequence of the modified 17 C-terminal residues is selected from SEQ. ID NO: 6, SEQ. ID NO: 7 or SEQ. ID NO: 8. In a related embodiment the present invention further describes an expression vector comprising a nucleic acid sequence that encodes a fibrinogen wherein the γ-chain comprises one or more modified 17 C-terminal residues, wherein the sequence of the modified 17 C-terminal residues is selected from SEQ. ID NO: 6, SEQ. ID NO: 7 or SEQ. ID NO: 8. In addition the sequences SEQ ID NO: 24-132 are also included herein and may be used in the present invention.

In yet another embodiment the present invention relates to a protein produced by a cell that encodes a nucleic acid that expresses the protein, comprising, a recombinant fibrinogen wherein the fibrinogen γ-chain comprises one or more modified 17 C-terminal residues, wherein the sequence of the modified 17 C-terminal residues is selected from SEQ. ID NO: 6, SEQ. ID NO: 7 or SEQ. ID NO: 8 and one or more N-terminal modified β-chains, wherein the sequence of the N-terminal modified β-chains comprises SEQ. ID NO: 14, SEQ. ID NO: 15, SEQ. ID NO: 16, SEQ. ID NO: 17, SEQ. ID NO: 20, SEQ. ID NO: 21, SEQ. ID NO: 22 or SEQ. ID NO: 23. In addition the sequences SEQ ID NO: 24-132 are also included herein and may be used in the present invention. The recombinant fibrinogen proteins of the present invention are expressed in a bacterial cell, a yeast cell, a mammalian cell or an animal.

### BRIEF DESCRIPTION OF THE SEVERAL VIEWS OF THE DRAWINGS

For a more complete understanding of the features and advantages of the present invention, reference is now made to the detailed description of the invention along with the accompanying figures and in which:

FIG. 1 shows the species specificity of ClfA:Fg binding. Recombinant ClfA_{327C/541C} does not bind sheep Fg. The disulfide mutant rClfA_{327C/541C} binds immobilized Fg from different animal species with different apparent affinities in a solid-phase assay;

FIG. 2 shows that soluble ClfA N2N3 (ClfA₂₂₉₋₅₄₅) can prevent binding of bacteria expressing ClfA (L. lactis ClfA) to the platelet integrin α_{IIb}β₃. L. lactis ClfA 5×10⁸ cfu/ml was incubated in the presence of 2 mg/ml human Fg and varying amounts of ClfA₂₂₉₋₅₄₅ N2N3 was added to microtiter plate coated with platelet integrin α_{IIb}β₃. Bound bacteria were detected using crystal violet;

FIGS. 3A and 3B show the isothermal calorimetry results for the binding of human (FIG. 3A) or sheep (FIG. 3B) 15-mer C-terminal Fg γ-peptides to ClfA₂₂₉₋₅₄₅ in solution;

FIG. 4 is a representation of the binding pockets formed between the N2 and N3 domains of Fbl and ClfA bound to human and sheep (ovine) Fg γ-chain peptide. Ribbon representation of the homology model of Fbl N2-N3 (pink) overlaid on to the crystal structure of ClfA (cyan):peptide (magenta) complex. Side chain atoms of both the residues Gln (yellow) of the human sequence (crystal structure) and Lys (blue) from the sheep sequence (model) at position 407 are shown as ball and stick objects. Thr 383 and backbone atoms of Ile 384 that could make severe steric clashes with Lys are shown in red. Residue numbers corresponding to Thr383 and Ile 384 of Fbl are shown in parenthesis. Fbl is a ClfA homolog from S. lugdunensis predicted to have similar overall structure as ClfA;

FIG. 5 is a graph of the binding of human fibrinogen to human platelet integrin α_{IIb}β₃ in presence of varying concentrations of human wild-type 15-mer peptide or Q407K peptide;

FIG. 6 is a plot that illustrates that MSCRAMM ClfA does not bind to salmon Fg; and

FIG. 7 is a plot demonstrating that salmon fibrinogen does not support adherence of S. aureus bacteria.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

The objective of the present invention is to make modified or isolate natural variants of fibrinogen and fibrinogen derived products lacking the bacterial adherence properties normally associated with fibrinogen. Despite the modification of the fibrinogen described herein, the fibrinogen of the present invention will function similarly in blood clotting, cross-linking, and platelet aggregation, i.e. normal biological function but deprived of bacterial binding. The major advantage of the new type of fibrinogen of the present invention is the reduction or even absence of bacterial adherence compared to normal fibrinogen, although being a fibrinogen with complete retention of normal physiological functions. The invention also contains modifications that will decrease platelet binding that can be combined with reduced bacterial attachment.

Fibrinogen and fibrinogen derived compounds are currently being used for a variety of medical conditions. Fibrin glue is one application where fibrinogen is currently used, especially in connection to surgery. Also, medical devices in contact with blood rapidly get a spontaneous coating of fibrinogen, leading to clumping/colonization of bacteria. Pre-coating of these devices with modified fibrinogen will prevent colonization from bacteria. The purpose of this invention is to have improved fibrinogen products that lack the ability to adhere to the pathogenic bacteria. The present invention therefore uses modified or isolated natural variants of fibrinogen (Fg) sequences to make new fibrinogen products. These new improved products can be used to prevent bacterial attachment or even treat infections caused by staphylococcal species from the following list: S. afermentans, S. aureus, S. auricularis, S. capitis, S. caprae, S. cohnii, S. epidermidis, S. felis, S. haemolyticus, S. hominis, S. intermedius, S. lugdunensis, S. pettenkoferi, S. saprophyticus, S. schleiferi, S. simulans, S. vitulus, S. warneri, and S. xylosus and streptococcal species including Streptococcus agalactiae (Group B Streptococcus) and Streptococcus pyogenes (Group A Streptococcus). It is also known that the binding between bacteria and fibrinogen is of importance for the virulence mechanisms of the pathogens¹. The modified fibrinogen and products thereof described herein can reduce bacterial binding and hence also the resulting virulence caused by a number of different bacterial pathogens may also be reduced.

The present invention features novel aspects and has a wide variety of applications in varied areas of healthcare. Fibrin glue or fibrin sealant is one example of a fibrinogen derived product commonly used in association with surgery. The present invention provides the development of recombinant, transgenic or isolated natural variants of fibrinogen or fibrinogen related products that are made into fibrin sealant/fibrin glue that prevent MSCRAMM:fibrinogen interactions but have little or no effect on FXIIIa cross-linking or binding to host proteins like α_{IIb}β₃ integrin or α_{M}β₂ integrin. The present invention provides the development of recombinant, transgenic or isolated natural variants of fibrinogen or fibrinogen related products that are made into modified fibrinogen for systemic use in humans (intravenous, subcutaneous or intraperitoneal administration) that will prevent MSCRAMM:fibrinogen interactions but have little or no effect on FXIIIa cross-linking or binding to host proteins like α_{IIb}β₃ integrin or α_{M}β₂ integrin. The present invention provides the development of recombinant, transgenic or natural variants of fibrinogen or fibrinogen related products coated onto medical devices that are in contact with human blood or fluids, such as various injection devices, valves and artificial medical components. The coating with the modified fibrinogen will prevent MSCRAMM:fibrinogen interactions thereby reducing the clumping/colonization of bacteria that is a common problem. Despite the modifications, the modified fibrinogen will have little or no effect on FXIIIa cross-linking or binding to host proteins like α_{IIb}β₃ integrin or α_{M}β₂ integrin. Further, the present inventors also suggest additional sequence modifications that can decrease binding and subsequent activation/aggregation of platelets to these devices, and (iv) development of recombinant or transgenic fibrinogen or fibrinogen related products that are hybrid molecules consisting of certain MSCRAMMs and the modified fibrinogen sequence. These hybrid molecules will bind to the host fibrinogen chains but will have a covalently linked extension of the described modified fibrinogen sequences that allow beneficial interactions with the host, such as α_{IIb}β₃ integrin or α_{M}β₂ integrin or even FXIIIa cross-linking Hence, the bacterial binding to the fibrinogen chains will be blocked, and bacterial pathogen adherence and virulence can be inhibited, although host interactions will still be functional through the modified fibrinogen sequence extension. These hybrid molecules may also be used in vaccine formulations.

Generation of a Three-dimensional Model of rFbl206-533: Molecular modeling studies were performed using InsightII software (Accrelys Inc.) A homology model of Fbl:Fg γ-chain peptide complex in closed conformation was generated using the crystal structure of ClfA:Fg γ-chain D410A peptide complex as template². Sequence alignment using LALIGN³ server showed no gaps in the sequences between ClfA and Fbl. A structure of the N2N3 subdomains of Fbl206-533 was modeled using MODELLER available in the HOMOLOGY module of InsightII using the sequence alignment from LALIGN. The wild type sequence of Fg peptide, rather than the D410A variation in the ClfA-peptide complex was used for modeling Fg γ-peptide. The resultant model did not show any steric violation between the Fg γ-peptide and Fbl. The stereochemical parameters of the model were checked using PROCHECK⁴, and figures with ribbon models were generated using RIBBONS⁵.

Synthesis of Peptides: Human 17-mer, human 15-mer and putative sheep (ovine) 15-mer were synthesized by Biomatik (Wilmington, Del.). The present invention includes the Human 17-mer (GEGQQHHLGGAKQAGDV) (SEQ. ID NO: 1) and Q407K (GEGQQHHLGGAKKAGDV) (SEQ. ID NO: 6). Peptide sequences described in the study include human 15-mer (GQQHHLGGAKQAGDV) (SEQ. ID NO: 4), and sheep 15-mer (GQQHHLGGAKKAGDV) (SEQ. ID NO: 5).

Microcalorimetry analysis of peptide binding to recombinant proteins: The binding between rClfA₂₂₉₋₅₄₅ and the human 17-mer, human 15-mer and sheep 15-mer were tested using isothermal titration calorimetry. Peptide 0.6-0.9 mM was injected into a cell with rClfA₂₂₉₋₅₄₅ in a VP-ITC (MicroCal Inc.). The resulting data was analyzed using Origin 5.0 from MicroCal using a single binding site model.

Recombinant protein expression: Recombinant ClfA₂₂₉₋₅₄₅ and ClfA_{327C/541C} were prepared as 6×His tagged proteins as previously described². Briefly, recombinant proteins were expressed in E. coli and purified using first a Ni²⁺-column followed by ion exchange Q-column and if necessary also purified on a gel filtration column.

L. lactis ClfA binding to platelet integrin through a Fg bridge: Integrin α_{IIb}β₃ was coated onto plastic microtiter plate. After blocking, the human Fg (2 mg/ml) in presence of L. lactis ClfA 5×10⁸ cfu/ml was added. To see if rClfA₂₂₉₋₅₄₅ could inhibit binding, varying amounts of rClfA₂₂₉₋₅₄₅ was added simultaneously with the L. lactis ClfA to inhibit the binding of bacteria to the integrin α_{IIb}β₃. Bound bacteria were quantified by crystal violet staining Recombinant ClfA₂₂₉₋₅₄₅ could efficiently block the binding of L. lactis ClfA to the platelet integrin.

Enzyme-linked Immunosorbent Assay (ELISA): The ability of the wild-type ClfA₂₂₉₋₅₄₅ and disulfide ClfA mutants to bind Fg was analyzed by ELISA-type binding assays. Immulon 4HBX Microtiter plates (Thermo) were coated with human Fg (1 μg/well) in HBS (10 mM HEPES, 100 mM NaCl, 3 mM EDTA, pH 7.4) over-night at 4° C. The wells were washed with HBS containing 0.05% (w/v) Tween-20 (HBST) and blocked with 5% (w/v) BSA in HBS for 1 hour at 25° C. The wells were washed 3 times with HBST and recombinant ClfA proteins in HBS were added and the plates were incubated at 25° C. for 1 hour. After incubation, the plates were washed 3 times with HBST. Anti-His antibodies (GE Healthcare) were added (1:3000 in HBS) and the plates were incubated at 25° C. for 1 hour. The wells were subsequently washed 3 times with HBST and incubated with Goat anti-mouse-AP secondary antibodies (diluted 1:3000 in HBS; Bio-Rad) at 25° C. for 1 hour. The wells were washed 3 times with HBST and AP-conjugated polyclonal antibodies were detected by addition of p-nitrophenyl phosphate (Sigma) in 1 M diethanolamine (0.5 mM MgCl₂, pH 9.8) and incubated at 25° C. for 30-60 min. The plates were read at 405 nm in a ELISA plate reader (Themomax, Molecular Devices).

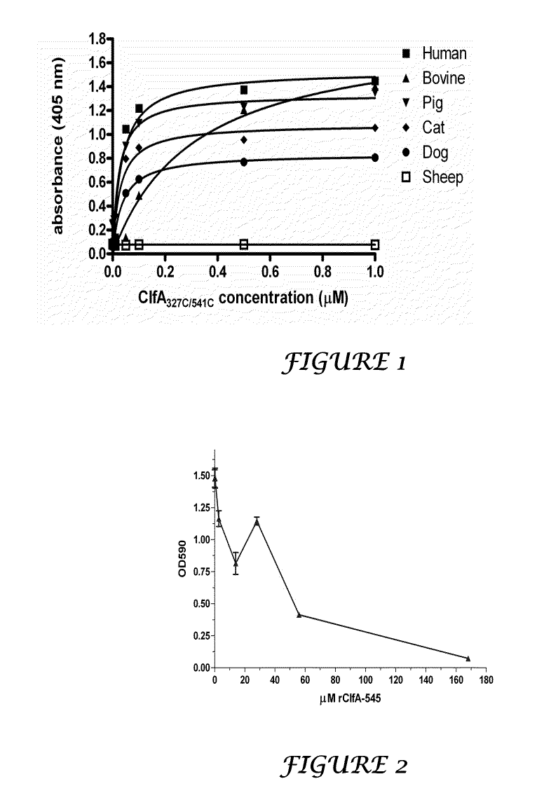

As shown in FIG. 1, disulfide bonded ClfA (ClfA_{327C/541C}) does not bind the ovine (sheep) Fg. However, Fg from most other species bound ClfA. To probe the differences in the sheep Fg compared to the human Fg a search for the ovine Fg γ-chain sequence was undertaken using public databases.

FIG. 2 shows that soluble ClfA N2N3 (ClfA₂₂₉₋₅₄₅) can prevent binding of bacteria expressing ClfA (L. lactis ClfA) to the platelet integrin α_{IIb}β₃. L. lactis ClfA 5×10⁸ cfu/ml was incubated in the presence of 2 mg/ml human Fg and varying amounts of ClfA₂₂₉₋₅₄₅ N2N3 was added to microtiter plate coated with platelet integrin α_{IIb}β₃. Bound bacteria were detected using crystal violet.

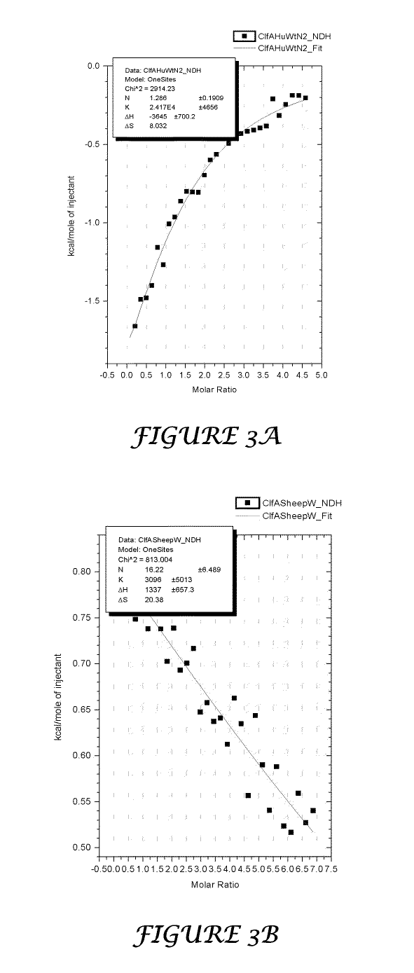

FIGS. 3A and 3B show the isothermal calorimetry results for the binding of human (FIG. 3A) or sheep (FIG. 3B) 15-mer C-terminal Fg γ-peptides to ClfA₂₂₉₋₅₄₅ in solution.

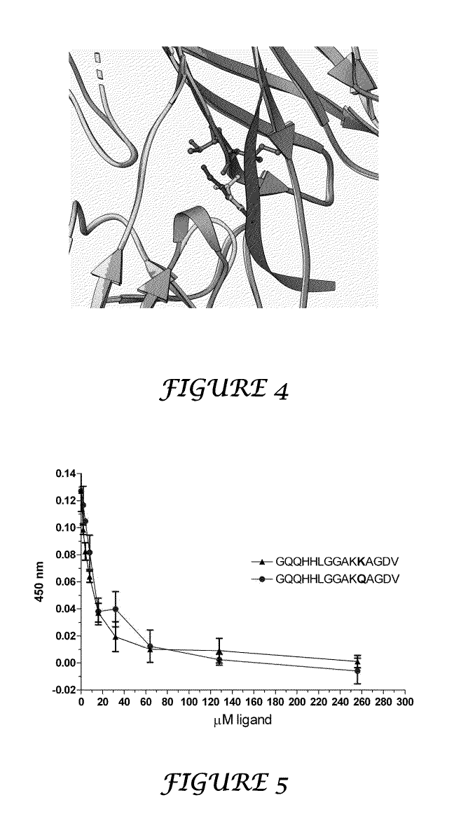

FIG. 4 is a representation of the binding pockets formed between the N2 and N3 domains of Fbl and ClfA bound to human and sheep (ovine) Fg γ-chain peptide. Ribbon representation of the homology model of Fbl N2-N3 (pink) overlaid on to the crystal structure of ClfA (cyan):peptide (magenta) complex. Side chain atoms of both the residues Gln (yellow) of the human sequence (crystal structure) and Lys (blue) from the sheep sequence (model) at position 407 are shown as ball and stick objects. Thr 383 and backbone atoms of Ile 384 that could make severe steric clashes with Lys are shown in red. Residue numbers corresponding to Thr383 and Ile 384 of Fbl are shown in parenthesis. Fbl is a ClfA homolog from S. lugdunensis predicted to have similar overall structure as ClfA;

The ovine Fg γ-chain is not annotated in the public databases, but a bioinformatic search for ovine EST clones with the BLAST (tblastn module)⁶ using the human C-terminal sequence (351-411) as template generated a liver cDNA sequence (GenBank gi 114476568) with a matching stop codon and 90% identity to the human C-terminal sequence. When the C-terminal sequences were aligned, the putative ovine sequence had an apparent gap at human position 395, but also substitutions E396D and Q407K when compared to the C-terminal human Fg γ 17-mer In order to determine if the Q407K variation is responsible for the inability of ClfA to bind ovine Fg, a peptide corresponding to the C-terminal 15 residues of the predicted ovine Fg γ-chain was compared to the equivalent human Fg 15-mer. ClfA₂₂₉₋₅₄₅ bound the human 15-mer peptide with a K_{D} of 40 μM and to the human 17-mer peptide with a K_{D} of 23 μM but the 15-mer sheep (ovine) peptide (SEQ. ID NO: 5) did not bind detectably to ClfA₂₂₉₋₅₄₅ (FIG. 3B). Crystal structure of the ClfA:peptide complex showed that Gln 407 of Fg makes key interactions with the ClfA and is also completely buried in the complex. A less bulky Ala substitution Q407A from the alanine scan studies done by the present inventors showed the Q to A variation affects binding but does not abolish binding to ClfA².

Visual examination of the model of the ClfA:peptide complex with a Q407K variation in the γ-chain peptide suggests that in the sheep Fg γ-chain sequence, the bulky residue K407 (human Fg numbering) clashes with Thr 383 and backbone atoms of Ile 384 of ClfA, preventing the sheep sequence to fit into the binding trench in addition to losing favorable interactions with ClfA (FIG. 4). In addition, a polar uncharged Gln to positively charged Lys could also pose charge-charge repulsion with any of the surrounding positively charged residues such as His 252 and Lys 381. Thus the loss of binding of ClfA to ovine Fg may be attributable to the Q407K substitution in the C-terminus of the sheep (ovine) Fg γ-chain.

Shown below are the wild-type γ-chain and modified γ-chain sequences with 1-4 amino acid changes. These modified sequences will not bind to ClfA or other homologous proteins that bind to Fg γ-chain in a similar mode of binding.

| | | |
|---|---|---|
| Human γ-chain | GEGQQHHLGGAKQAGDV | (SEQ. ID NO: 1) |
| Modified γ | GEGQQHHLGGAKKAGDV | (SEQ. ID NO: 6) |
| Modified γ | GEGQQHHLGGAARAGDV | (SEQ. ID NO: 7) |
| Modified γ | GEGQQHHLGGAKHAGDV | (SEQ. ID NO: 8) |

In the sequences described above the Q to K substitution may also be substituted to other positively charged amino acids like R or H. In addition the sequences SEQ ID NO: 24-132 are also included herein and may be used in the present invention.

As an alternate strategy also amino acids or modifications of amino acids at Q407 can be tested (except A) since it is known that this substitution will not have a dramatic effect on binding to ClfA². The present inventors solved the three-dimensional structure of the apo-protein and ligand bound form of ClfA. A homology model of Fbl was built based on its amino acid sequence similarity and structural similarity to ClfA. Amino acid residues in the ligand binding trench are completely conserved in contrast to residues located elsewhere on the protein. Overall it appeared that ClfA and Fbl are very similar.

The inventors further describe a N-terminal sequence modification of the Fg β-chain that abolishes binding to S. epidermidis SdrG and any other homologous MSCRAMMs that target the N-terminus β-chain in a similar mode of binding. It should be noted that it is not a problem to combine several proposed mutations into the same Fg molecule to make a Fg with significantly reduced binding to several Staphylococcal or Streptococcal species, but retaining essential biological functions.

Previously reported data have shown that some residues are critical for SdrG binding⁷. Critical residues in Fg for SdrG binding are underlined: NEEGFFSA [symbol] GHRPLD (SEQ. ID NO: 9). Destroying the interaction of any one of the amino acids FFSAR (SEQ. ID NO: 10) significantly (70-90%) reduced binding to SdrG⁷. The arginine ( [symbol] ) is critical for the thrombin activity and can not be changed in order for it not to affect thrombin activity. Changing F, F, S to less bulkier alanines (A) destroys the side chain interaction for each of the F, F and S residues. Similarly changing each of the F, F, S to glycines would affect the binding to SdrG. The resultant sequence when FFS changes to GGG would look like GGGAR (SEQ. ID NO: 11) which is nothing but a part of the Fg α-chain sequence recognized by thrombin.

This design of the new sequence of β-chain also corroborates the known fact that thrombin cleaves both the Fg α- and β-chains, whereas SdrG selectively binds to the β-chain⁸. Therefore, by making a modified β-chain that in part mimics the α-chain, the modified Fg β-chain will no longer bind SdrG, but will be cleaved by thrombin and therefore not affect normal function.

Shown below are modified β-chain sequences with 1-4 amino acid changes. Enzyme nomenclature:

| | | |
|---|---|---|
| Human α | GGGV [symbol] ↓GPRVVE | (SEQ. ID NO: 12) |
| Human β | GFFSA [symbol] ↓GHRPL | (SEQ. ID NO: 13) |
| modified β | GFF [symbol] A [symbol] ↓GHRPL | (SEQ. ID NO: 14) |
| | 1 change | |
| modified β | GF [symbol] A [symbol] ↓GHRPL | (SEQ. ID NO: 15) |
| | 2 changes | |
| modified β | G [symbol] A [symbol] ↓GHRPL | (SEQ. ID NO: 16) |
| | 3 changes | |
| modified β | G [symbol] ↓GHRPL | (SEQ. ID NO: 17) |
| | 4 changes | |

P4-P3-P2-P1↓P1′-P2′-P3′
(↓ = Scissile bond)

As an alternate strategy, S to D instead of S to G could also be tested. Hirsch et al.⁹ lists a number of proteins that are cleaved by thrombin; including bovine (LDP [symbol] ↓IVDG) (SEQ. ID NO: 18) and human protein C (VDP [symbol] ↓LIDG) (SEQ. ID NO: 19). It is evident that aspartic acid (D) at position P3 (See position 3 in the alignment below) is compatible with thrombin activity. Therefore as an alternative, these sequences are of interest for reduced SdrG binding but retained thrombin cleavage activity: Enzyme nomenclature:

| | | |
|---|---|---|
| Position | 3 2 1↓ | |
| modified β | GFF [symbol] A [symbol] ↓GHRPL | (SEQ. ID NO: 20) |
| | 1 change | |
| modified β | GF [symbol] A [symbol] ↓GHRPL | (SEQ. ID NO: 21) |
| | 2 changes | |
| modified β | G [symbol] A [symbol] ↓GHRPL | (SEQ. ID NO: 22) |
| | 3 changes | |
| modified β | G [symbol] ↓GHRPL | (SEQ. ID NO: 23) |
| | 4 changes | |

P4-P3-P2-P1↓P1′-P2′-P3′
(↓ = Scissile bond)

The sequence that shows high selectivity towards thrombin and loss of binding to SdrG can be used to make modifications in Fg. In addition the sequences SEQ ID NO: 24-132 are also included herein and may be used in the present invention.

The present inventors further demonstrated that ClfA N2N3 can inhibit bacteria expressing ClfA to adhere to Fg coated to a plastic plate. As shown in FIG. 2 recombinant ClfA₂₂₉₋₅₄₅ N2N3 can efficiently block adherence of L. lactis ClfA to platelet integrin α_{IIb}β₃ using a Fg bridge. Based on this finding, a set of modified hybrid molecules are proposed that will inhibit bacteria to use Fg as a bridge, but will still be able to mediate binding to platelet integrin that is necessary for normal physiological functions in the body. Specifically N- and C-terminal extensions are proposed. As demonstrated in the examples below.

ClfA N2N3 domain with a C-terminal linker followed by a sequence containing SEQ. ID NO: 6, SEQ. ID NO: 7 or SEQ. ID, NO: 8 or a variant thereof. In addition the sequences SEQ ID NO: 24-132 are also included herein and may be used in the present invention. This hybrid molecule will bind the human Fg γ-chain C-terminus and prevent binding by S. aureus ClfA and other bacterial proteins binding in a similar mode as ClfA. However, the SEQ. ID NO: 6, SEQ. ID NO: 7 or SEQ. ID, NO: 8 or variants thereof will still allow binding to host molecules like platelet and leukocyte integrins. Since the integrins can still function with this new Fg γ-chain sequence, the patient can be given a significantly higher dose of this hybrid molecule compared to just the ClfA N2N3 alone. Since ClfA N2N3 is a platelet inhibitor¹⁰ high doses of this protein alone would have side effects like bleedings. Instead of ClfA N2N3 a closed cysteine mutant with stronger binding to Fg may be used instead².

SdrG N2N3 domain with a N-terminal linker followed by a sequence containing SEQ. ID NO: 14, SEQ. ID NO: 15 or SEQ. ID NO: 16 or variants thereof. This hybrid will bind the human Fg β N-terminus and prevent binding by S. epidermidis SdrG and other bacterial proteins binding in a similar mode as SdrG. However the SEQ. ID NO: 14, SEQ. ID NO: 15 or SEQ. ID NO: 16 or variants thereof will still allow cleavage by thrombin so that fibrinopeptides can be cleaved off to perform their physiological functions.

The claims, in general, refer to recombinant or transgenic Fg, and includes isolated natural variants of Fg from humans. Isolated natural variants may be used in stopping or preventing blood loss in patients with wounds, for example in emergency settings, including but not limited to blood loss in the battle field or traffic accidents. Donor blood/plasma from persons with isolated natural variants may be beneficial as transfusion material in circumstances where risks of infection are increased.

The presence of natural variants of Fg can be identified using various methods. These could be identified by isolating donated plasma/blood or Fg and subsequently testing it in enzyme-linked immunosorbent assays or other biochemical methods for MSCRAMM adherence. Other methods of identification include, but are not limited to, genotyping the Fg α-, β- and γ-genes of these persons to identify natural variants. Genotyping followed by molecular modeling will then identify persons with mutations that are not capable of binding to the MSCRAMMs of interest.

After identification of either donors or alternatively after establishing plasma or Fg samples with lowered binding to Fg, Fg from these persons can be pooled and processed further. It is expected that a number of these persons will be heterozygous for the mutations in their Fg genes. It is therefore possible that further enrichment of the Fg may be necessary for making the infection-resistant product. Bacterial MSCRAMMs can be conjugated to various matrices and used to trap the fraction of Fg that still binds to these MSCRAMMs or even killed bacterial pathogens can be used to trap Fg that binds to the pathogen.

FIG. 5 is a graph of the absorbance of Human wild-type 15-mer peptide and Q407K peptide. A 15-mer peptide with the Q407K substitution still binds to the platelet integrin α_{IIb}β₃ similar to the wild-type 15-mer. Human platelet integrin α_{IIb}β₃ was coated onto plastic microtiter plates and blocked with bovine serum albumin and 10 nM Fg was added in presence of varying concentrations of peptide. Bound Fg was detected using goat anti-human Fg antibodies and secondary rabbit antibodies against goat conjugated to horse radish peroxidase (HRP) were used. Upon addition of reagent, the HRP color development was read in a microtiter plate reader at 450 nm. Triplicate samples per data point (one outlier data point omitted regarding the human peptide). Human wild-type 15-mer peptide (blue circles) and Q407K peptide (black triangles).

The Q407K substitution completely lost its binding to ClfA, in contrast to the wild-type peptide that binds to ClfA (FIGS. 3A and 3B). Since the Fg γ-chain is a biological hotspot it is important to establish that variant Fg sequences that will not bind to MSCRAMMs like ClfA may still bind to host molecules. Therefore the binding to platelet integrin α_{IIb}β₃ was tested in an inhibition assay (FIG. 5). The results show that the Q407K peptide inhibits binding of Fg to the platelet integrin as well as the wild-type peptide, thereby the Q407K mutation will functionally work equally well as the wild-type in normal haemostasis and clotting.

In addition, the attached sequences in the sequence listing described sequences that have reduced binding to ClfA. Other application of the present invention include, designer fibrinogen as a coating of orthopedic and dental implants (e.g., fibrinogen has been shown to be able to coat to implants such as titanium screws and can then be used for drug delivery that leads to faster adhesion to bone) and as a drug delivery vehicle (e.g., to use Fg-based materials for drug delivery).

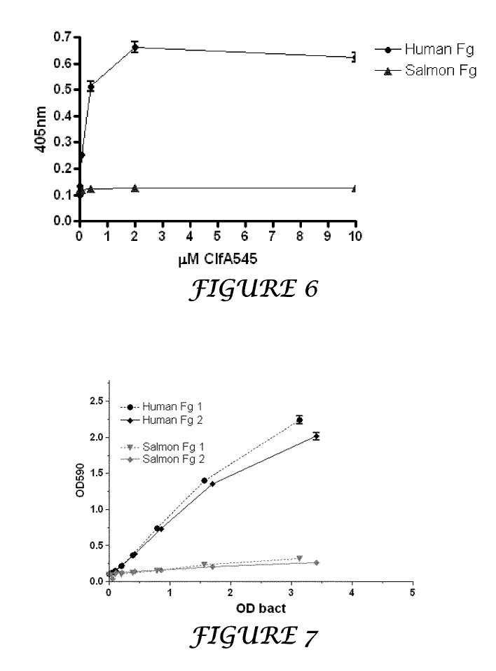

FIG. 6 is a plot that illustrates MSCRAMM ClfA does not bind to salmon Fg. To see if Fg from other species had reduced binding to ClfA₂₂₉₋₅₄₅, salmon and human Fg were coated onto plastic microtiter plates. Recombinant His-tagged ClfA N2N3 showed a dose-dependent binding to human Fg, whereas ClfA did not bind salmon Fg. Since the structure of ClfA in complex with a synthetic peptide mimicking the Fg γ C-terminus is available, it was possible to model the differences in amino acids to see possible explanations for loss of binding. Although the salmon (Salmo salar) Fg γ-chain gene is not annotated there is an EST (expressed sequence tag) with a similar sequence to the human Fg γ-chain (GenBank: BG934784.1). When this mRNA sequence was translated into the different reading frames one reading frame was very similar to the human Fg γ-chain, see alignment below. With this alignment, modeling displayed that the F at position 408 (bold, underlined number from human numbering) found in salmon Fg would not be well tolerated for binding to ClfA, which gives a theoretical explanation for the observed results regarding the binding. Alignment between C-terminus of human Fg γ-chain (huFg) and predicted salmon Fg γ-chain (saFg):

| | |
|---|---|
| huFg | |
| MKIIPFNRLTIGEGQQHHLGGAKQAGDV | SEQ ID NO: 133 |
| SaFg | |
| MKIIPTNRITAGDGQQT----GGVKQFGGLGDN | SEQ ID NO: 134 |

This suggests that sequence variation also at this position 408 can lead to reduced binding to ClfA and can be explored in isolation of natural human variants or in recombinant or transgenic fibrinogen. Additionally there is also a mismatch in the N-terminal end of the ClfA binding region, 400HHL403 due to the gap in the amino acid sequence in this region. Therefore in addition to the negative effect played by A408F, the substitutions H400Q, H401Q and L402T may also contribute to the reduced binding.

About 3 mg lyophilized salmon Fg (batch “A”) was dissolved into 3 mg/ml in PBS. Human and salmon Fg was coated onto 4HBX microtiter plates (1 μg/well) at 4° C. over-night in Dulbecco's PBS without MgCl₂ nor CaCl₂ ions). The plate was blocked for 1 hour room temperature (RT) using 5% bovine serum albumin (BSA) in TBS-T. Recombinant His-tagged ClfA₂₂₉₋₅₄₅ was added in varying concentrations to test for binding to Fg using TBS solution for 1 hour at room temperature. Bound ClfA was detected with anti-ClfA rabbit antibodies (dil 1:2000) in TBS-T with 1% BSA followed by goat anti-rabbit secondary antibodies (dil 1:2000) conjugated to alkaline phosphatase using the same buffer. Color detection was read after 30-60 min at 405 nm in a microtiter plate reader using phospatase substrate from Sigma dissolved in 1M DEA. No background subtraction, data points are triplicates except for no ClfA where six data points were used. Human or salmon Fg was coated over-night and after blocking varying concentrations of ClfA was added to Fg. Bound ClfA was detected as described in the Methods section.

FIG. 7 is a plot of salmon fibrinogen adherence of S. aureus bacteria. S. aureus USA300 NARSA was grown in tryptic soy broth over-night and the bacterial binding to human and salmon fibrinogen was tested. Whereas S. aureus USA300 bound human fibrinogen, essentially no binding was seen to salmon Fg. Salmon fibrinogen does not support the adherence of S. aureus bacteria. Salmon fibrinogen is being investigated for use as a biomaterial. Efforts were taken to determine if S. aureus could attach to salmon fibrinogen. S. aureus USA300 NARSA is a methicillin-resistant S. aureus (MRSA) strain that has the genes for a number of MSCRAMMs, including Fg-binding MSCRAMMs. S. aureus USA300 NARSA was grown in tryptic soy broth over-night and the bacterial binding to human and salmon fibrinogen was tested. Whereas S. aureus USA300 bound human fibrinogen, essentially no binding was seen to salmon Fg (FIG. 7). There are at least two important implications of this finding: First, none of the Fg-binding MSCRAMMs in S. aureus USA300 NARSA recognize salmon Fg, meaning that the use of salmon Fg-derived products in humans (or other species) are likely to bind less if not at all to S. aureus and possibly also other bacterial pathogens. Second, if the binding site is identified in human (or other species) Fg for binding to a particular MSCRAMM and the said MSCRAMM cannot bind to salmon Fg, the human (or other species) Fg sequence can be mutated in the binding region so that the said MSCRAMM no longer can bind the modified Fg. The mutation may or may not be identical to the salmon Fg sequence. If the salmon sequence is used, this method provides a rapid way of generating a biologically active Fg with essentially no bacterial binding, since salmon Fg has biological clotting activity. This novel Fg can be produced using recombinant or transgenic methods.

Microtiter plates were coated with 1 μg human or salmon Fg per well over-night at 4° C., followed by blocking with 5% bovine serum albumin in TBS-T (Tris-buffered saline with 0.05% Tween-20) in room temperature for about 1 hour. Two bacterial colonies (designated by 1 or 2 in FIG. 7) of S. aureus USA300 NARSA (an MRSA strain) were inoculated in tryptic soy broth at 37° C. over-night, washed and resuspended in PBS and various bacterial dilutions were incubated in the wells for 1 hour at room temperature. After washing in PBS, bound bacteria was fixed using 25% formaldehyde followed by 0.5% crystal violet stain. After PBS washing, 10% acetic acid was added and bound color was measured by crystal violet staining and optical density read at 590 nm in a microtiter plate reader.

FIG. 7 illustrates that S. aureus does not adhere to salmon fibrinogen. Bacterial suspensions were added to human or salmon fibrinogen coated microtiter plates. Adherence was quantified by crystal violet staining and optimal density was read at 590 nm. The results indicate that salmon fibrinogen does not support S. aureus adherence.

It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, MB, BBC, AAABCCCC, CBBAAA, CABABB, and so forth. The skilled artisan will understand that typically there is no limit on the number of items or terms in any combination, unless otherwise apparent from the context.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

### REFERENCES

U.S. Pat. No. 6,177,084: S. aureus fibrinogen binding protein.
U.S. Pat. No. 6,037,457: Method for recombinant fibrinogen production.
1. Rivera, J., G. Vannakambadi, et al. (2007). “Fibrinogen-binding proteins of Gram-positive bacteria.” Thromb Haemost 98(3): 503-11.
2. Ganesh, V. K., J. J. Rivera, et al. (2008). “A structural model of the Staphylococcus aureus ClfA-fibrinogen interaction opens new avenues for the design of anti-staphylococcal therapeutics.” PLoS Pathog 4(11): e1000226.
3. Huang, X. M., W. (1991). “A time-efficient, linear-space local similarity algorithm.” Adv. Appl. Math 12: 337-357.
4. Laskowski, R. A., D. S. Moss, et al. (1993). “Main-chain bond lengths and bond angles in protein structures.” J Mol Biol 231(4): 1049-67.
5. Carson, M. (1997). “Ribbon models for macromolecules.” J Mol Graph 5: 103-106.
6. Altschul, Stephen F., Warren Gish, Webb Miller, Eugene W. Myers, and David J. Lipman (1990) “Basic local alignment search tool.” J. Mol. Biol. 215:403-10.
7. Ponnuraj, K., M. G. Bowden, et al. (2003). “A “dock, lock, and latch” structural model for a staphylococcal adhesin binding to fibrinogen.” Cell 115(2): 217-28.
8. Davis, S. L., S. Gurusiddappa, et al. (2001). “SdrG, a fibrinogen-binding bacterial adhesin of the microbial surface components recognizing adhesive matrix molecules subfamily from Staphylococcus epidermidis, targets the thrombin cleavage site in the Bbeta chain.” J Biol Chem 276(30): 27799-805.
9. Colman, R. W., J. Hirsh, et al. (1994). Hemostasis and Thrombosis. Philadelphia, J. B. Lippincott Company.
10. Liu, C. Z., M. H. Shih, et al. (2005). “ClfA(221-550), a fibrinogen-binding segment of Staphylococcus aureus clumping factor A, disrupts fibrinogen function.” Thromb Haemost 94(2): 286-294.

What is claimed is:

1. A recombinant, transgenic or natural variant fibrinogen protein comprising
a fibrinogen wherein the γ-chain comprises one or more modified 17 C-terminal residues, wherein the sequence of the modified 17 C-terminal residues is selected from SEQ. ID NO: 6, SEQ. ID NO: 7, SEQ. ID NO: 8, and SEQ. ID NO: 24-134.

2. The recombinant, transgenic or natural variant fibrinogen protein of claim 1, wherein the fibrinogen protein is a human fibrinogen protein.

3. The recombinant, transgenic or natural variant fibrinogen protein of claim 1, wherein the fibrinogen protein is adapted for therapeutic use as a nasal spray, a foam, an eye drop, or a topical wound dressing in a pharmaceutically acceptable excipient.

4. The recombinant, transgenic or natural variant fibrinogen protein of claim 1, wherein the fibrinogen protein is non-ovine.

5. The recombinant, transgenic or natural variant fibrinogen protein of claim 1, wherein the fibrinogen β-chain comprises an N-terminal modified β-chain, wherein the sequence of the N-terminal modified β-chains is selected from SEQ. ID NO: 14, SEQ. ID NO: 15, SEQ. ID NO: 16, SEQ. ID NO: 17, SEQ. ID NO: 20, SEQ. ID NO: 21, SEQ. ID NO: 22 or SEQ. ID NO: 23.

6. A recombinant, transgenic or natural variant fibrinogen protein comprising a non-ovine fibrinogen and an ovine γ-chain sequence.

7. A fibrin glue or sealant composition comprising:
a first container comprising a fibrinogen wherein the γ-chain comprises one or more modified 17 C-terminal residues, wherein the sequence of the modified 17 C-terminal residues is selected from SEQ. ID NO: 6, SEQ. ID NO: 7 SEQ. ID NO: 8, and SEQ. ID NO: 24-134; and
a second container comprising a thrombin or one or more proteinases or enzymes, wherein a combination of the contents of the first and second containers form a fibrin clot.

8. The composition of claim 7, wherein the one or more proteinases are selected from the group consisting of batroxobin, okinaxobin, flavoxobin, arvin, cathepsin, or any combinations thereof.

9. The composition of claim 7, wherein the fibrinogen is non-ovine.

10. The composition of claim 7, wherein the fibrinogen protein is a human fibrinogen protein.

11. The composition of claim 7, wherein the composition further comprises coagulation Factor XIII.

12. The composition of claim 7, wherein the composition further comprises an antifibrinolytic agent.

13. The composition of claim 7, wherein the antifibrinolytic agent is ε-aminocaproic acid, p-aminomethylbenzoic acid or aprotinin.

14. A bandage comprising:
a backing comprising a paper, a cloth, a plastic, an open-cell polyurethane foam, an open-cell polyethylene foam, a nonwoven fabric; and
a fibrinogen wherein the γ-chain comprises one or more modified 17 C-terminal residues, wherein the sequence of the modified 17 C-terminal residues is selected from SEQ. ID NO: 6, SEQ. ID NO: 7, SEQ. ID NO: 8 and SEQ. ID NO: 24-134.

15. The bandage of claim 14, further comprising a carrier selected from a polymer thickener, water, preservatives, active surfactants or emulsifiers, antioxidants, sunscreens, or combinations thereof.

16. The bandage of claim 14, further comprising an adhesive or an adhesive layer on the backing.

17. The bandage of claim 14, wherein the carrier further comprises antimicrobial agents, anti-inflammatory agents, antiviral agents, local anesthetic agents, corticosteroids, destructive therapy agents, antifungals, antiandrogens, mild surfactants, and combinations thereof.

18. The bandage of claim 14, wherein the fibrinogen protein is a human fibrinogen protein.

19. A recombinant, transgenic or natural variant fibrinogen protein comprising:
a fibrinogen wherein a Staphylococcus sp. or a Streptococcus sp. bacterium does not attach to the γ-chain sequence of fibrinogen that comprises a modified 17 C-terminal residues, wherein the sequence of the modified 17 C-terminal residues is selected from SEQ. ID NO: 6, SEQ. ID NO: 7, SEQ. ID NO: 8, and SEQ. ID NO: 24-134.

20. A method of treating wound infections or promoting wound healing comprising the steps of:
identifying a patient in need of treatment against wound infections or promotion of wound healing; and
applying a fibrin sealant, a fibrin glue or a bandage, wherein the fibrin sealant, the fibrin glue or the bandage comprises a fibrinogen wherein the γ-chain comprises one or more modified 17 C-terminal residues, wherein the sequence of the modified 17 C-terminal residues is selected from SEQ. ID NO: 6, SEQ. ID NO: 7, SEQ. ID NO: 8, and SEQ. ID NO: 24-134.

21. The method of claim 20, wherein the fibrinogen protein is a human fibrinogen protein.

22. The method of claim 20, wherein the fibrinogen protein is a non-ovine fibrinogen comprising an ovine γ-chain sequence.

23. A method of treating sepsis, hypofibrinogenemia or congenital afibrinogenemia in a patient comprising the step of:
identifying a patient in need for treatment against sepsis, hypofibrinogenemia or congenital afibrinogenemia; and
systemically administering a fibrinogen wherein the γ-chain comprises one or more modified 17 C-terminal residues, wherein the sequence of the modified 17 C-terminal residues is selected from SEQ. ID NO: 6, SEQ. ID NO: 7, SEQ. ID NO: 8, and SEQ. ID NO: 24-134.

24. The method of claim 23, wherein the fibrinogen protein is a human fibrinogen protein.

25. The method of claim 23, wherein the fibrinogen protein is a non-ovine fibrinogen comprising an ovine γ-chain sequence.

26. A biodegradable polymer or matrix comprising:
a polymer or matrix; and
a fibrinogen in, on or about the polymer or matrix, wherein the γ-chain comprises one or more modified 17 C-terminal residues, wherein the sequence of the modified 17 C-terminal residues is selected from SEQ. ID NO: 6, SEQ. ID NO: 7, SEQ. ID NO: 8, and SEQ. ID NO: 24-134.

27. The polymer or matrix of claim 26, wherein the fibrinogen protein is a human fibrinogen protein.

28. The polymer or matrix of claim 26, wherein the fibrinogen protein is a non-ovine fibrinogen comprising an ovine γ-chain sequence.

29. A coated medical device, injection device, artificial valve or any medical component in contact with blood comprising a fibrinogen wherein the γ-chain comprises one or more modified 17 C-terminal residues, wherein the sequence of the modified 17 C-terminal residues is selected from SEQ. ID NO: 6, SEQ. ID NO: 7, SEQ. ID NO: 8, and SEQ. ID NO: 24-134.

30. The device of claim 29, wherein the fibrinogen protein is a human fibrinogen protein.

31. The device of claim 30, wherein the fibrinogen protein is a non-ovine fibrinogen comprising an ovine γ-chain sequence.

32. A method of preventing platelet adherence, bacterial adherence on a medical device, an injection device, an artificial valve or any medical component in contact with blood, comprising the step of:
coating or coupling the medical device, the injection device, the artificial valve or any medical component in contact with blood with a fibrinogen comprising an γ-chain comprises one or more modified 17 C-terminal residues selected from SEQ. ID NO: 6, SEQ. ID NO: 7, SEQ. ID NO: 8, and SEQ. ID NO: 24-134.

33. The method of claim 32, wherein the one or more synthetic fibrinogen protein are coated or coupled by using one or more chemical-cross linkers.

34. A method for preventing intravascular thrombosis comprising the step of coating or coupling a medical device, a injection device, a artificial valve or any medical component in contact with blood with a fibrinogen wherein the γ-chain comprises one or more modified 17 C-terminal residues, wherein the sequence of the modified 17 C-terminal residues is selected from SEQ. ID NO: 6, SEQ. ID NO: 7, SEQ. ID NO: 8, and SEQ. ID NO: 24-134.

35. The method of claim 34, wherein the coating prevents the adherence of platelets to the medical device, the injection device, the artificial valve or any medical component in contact with blood.

36. A composition comprising:
a fibrinogen wherein the γ-chain comprises one or more modified 17 C-terminal residues, wherein the sequence of the modified 17 C-terminal residues is selected SEQ. ID NO: 6, SEQ. ID NO: 7, SEQ. ID NO: 8, and SEQ. ID NO: 24-134; and
one or more optional vaccine adjuvants or excipients.

37. A pharmaceutical formulation comprising:
a fibrinogen wherein the γ-chain comprises one or more modified 17 C-terminal residues, wherein the sequence of the modified 17 C-terminal residues is selected from SEQ. ID NO: 6, SEQ. ID NO: 7, SEQ. ID NO: 8, and SEQ. ID NO: 24-134 disposed in one or more pharmaceutically acceptable excipients.

38. A nucleic acid comprising a sequence that encodes a fibrinogen wherein the γ-chain comprises one or more modified 17 C-terminal residues, wherein the sequence of the modified 17 C-terminal residues is selected from SEQ. ID NO: 6, SEQ. ID NO: 7, SEQ. ID NO: 8, and SEQ. ID NO: 24-134.

39. An expression vector comprising a nucleic acid sequence that encodes a fibrinogen wherein the γ-chain comprises one or more modified 17 C-terminal residues, wherein the sequence of the modified 17 C-terminal residues is selected from SEQ. ID NO: 6, SEQ. ID NO: 7, SEQ. ID NO: 8, and SEQ. ID NO: 24-134.

40. A protein produced by a cell that encodes a nucleic acid that expresses the protein, comprising:
a recombinant fibrinogen wherein the fibrinogen γ-chain comprises one or more modified 17 C-terminal residues, wherein the sequence of the modified 17 C-terminal residues is selected from SEQ. ID NO: 6, SEQ. ID NO: 7, SEQ. ID NO: 8, and SEQ. ID NO: 24-134; and
one or more N-terminal modified β-chains, wherein the sequence of the N-terminal modified β-chains comprises SEQ. ID NO: 14, SEQ. ID NO: 15, SEQ. ID NO: 16, SEQ. ID NO: 17, SEQ. ID NO: 20, SEQ. ID NO: 21, SEQ. ID NO: 22 or SEQ. ID NO: 23.

41. The protein of claim 40, wherein the recombinant fibrinogen proteins is expressed in a bacterial cell, a yeast cell, a mammalian cell or an animal.
